(19) United States
(12) Patent Application Publication
(10) Pub. No.: US 2023/0165654 A1
(43) Pub. Date: Jun. 1, 2023

(54) HOLDING DEVICE FOR A STERILE PRODUCT, STERILE PRODUCT, AND METHOD FOR MONITORING A STERILE PRODUCT CYCLE

(71) Applicant: Aesculap AG, Tuttlingen (DE)

(72) Inventors: Stephanie Auber, Tuttlingen (DE); Lukas Böhler, Furtwangen (DE); Roland-Alois Högerle, Tuttlingen (DE); Frederick Lenzenhuber, Tuttlingen (DE); Ralf Pfister, Trossingen (DE)

(73) Assignee: Aesculap AG, Tuttlingen (DE)

(21) Appl. No.: 17/920,924

(22) PCT Filed: Apr. 27, 2021
(86) PCT No.: PCT/EP2021/060926

§ 371 Date: Oct. 24, 2022

(30) Foreign Application Priority Data
Apr. 29, 2020 (DE) 10 2020 111 712.4

(51) Int. Cl. A61B 50/39; A61L 2/28

(52) U.S. Cl. CPC A61B 50/39; A61L 2/28; A61L 2202/12; A61L 2202/14; A61L 2202/21

(57) ABSTRACT

A holding device for a sterile product, in particular for a sterile product container or a soft packaging, which device is provided to hold, during a preparation process, a medical product to be sterilised, and comprises communication means, and a part of the communication means is designed for a frequency range such that the part, when the sterile product is closed, supplies data in relation to the medical product to an environment of the sterile product. Also provided are a sterile product and a method for monitoring a sterile product cycle.

The invention relates to a holding device for sterile products, in particular for a sterile product container or a soft packaging, wherein sterile product shall also mean a sterile container with an inserted screen basket as well as screen baskets in soft packaging, and to a method for monitoring a sterile product cycle. The holding device may in particular be provided to hold at least one sterilisable medical product, in particular during a preparation process (including ultrasonic cleaning, cleaning and disinfection, maintenance, e.g. lubricating and/or sterilisation) and to store the medical product before and/or after the preparation process.

### BACKGROUND OF THE INVENTION

The detection of a sterile product cycle for medical products, such as medical-technical products, for instance, tools (application parts, tools, etc.), surgical instruments or handpieces, is mostly based on written documentation or checks at the end of a preparation cycle. The problem involved is that one cannot say with certainty how pre-cleaning, cleaning and disinfection, maintenance and sterilisation were performed. Although valid data are produced by hand-written records, this procedure is error-prone. Furthermore, service times of electrical, pneumatic, or hydraulic products cannot be documented in a manner related to the product (individual product level). A further problem is the lacking identification of individual products and the detection of their lifetime: catchword traceability.

There is no method of detecting the entire sterile product cycle along with the use. The acquiring of sensor and transponder data is limited to a product-related holder. Only few data about the medical products (e.g. motors, handpieces, surgical instruments, etc.) are acquired and thus limit the possibilities of evaluation and of triggering further processes such as services, replacement deliveries, etc.

Holding devices of the present type are provided inter alia for the cleaning and holding of surgical handpieces and are described inter alia in DE 10 2010 017 624. The holders are arranged with medical products arranged in the holders in medical cleaning equipment, in particular sterilisers, rinsing plants, or the like. Moreover, there is the possibility that the holders may be used along with sterile product containers and be arranged within same, so that it is ensured after a sterilisation of the medical products that a new contamination of the medical products is efficiently prevented.

Moreover, it is also usual that, for instance, reusable implants are reprocessed in an analogous manner as the afore-mentioned medical products. In a plurality of these reprocessable implants, in particular if they are manufactured of titanium, frequent reprocessing results in quality losses of the medical product. With titanium screws it is, for instance, the case that the thickness of the oxide layer protecting the titanium screw from corrosion and the formation of biofilm is reduced by the frequent use of aggressive cleaning substances. Reprocessable implants are frequently available in sets which, independently of whether individual elements of the set are actually used, always undergo a preparation process jointly.

### State of the Art

From the state of the art, for instance, in accordance with relevant products of the present applicant itself, holders of the afore-described type are known, which accompany the medical products held therein during the entire cleaning and preparation process. Such a holder is, for instance, known from DE 10 2010 017 624.

In order to be able to trace how often a medical product has been reprocessed during its lifetime it is, for instance, known to provide temperature sensors integrated in a motor of the medical product with their own energy supply units or energy harvesting units which detect the temperature during the preparation process and store it in a data storage of the motor. A motor comprising storage devices suited for this purpose is, for instance, described in DE 10 2011 050 192. Here, it is of particular disadvantage that such a solution4 takes a comparatively large installation space in the medical product and increases the weight thereof. Specifically with medical products applied in the field of neurosurgery, in minimally invasive methods, or the like these disadvantages are not acceptable.

In the monitoring of medical products it has turned out to be important that
a change in volume and/or weight of the medical product itself is preferably avoided, and
a monitoring of individual medical products is enabled.

Moreover, it would be desirable that
the acquired data can be read out wirelessly,
the acquired data can be supplied to an evaluation, and
the data can be accessed world-wide.

### Brief Description of the Invention

It is therefore an object of the invention to avoid or at least mitigate the disadvantages of the state of the art. In particular the sterile product cycle is to be improved with regard to traceability of the medical products and documentation with respect to the lifetime.

In accordance with the disclosure this object is solved in that a holding device for medical products mounted on the holding device during the sterile product cycle is provided. In accordance with the disclosure the holding device comprises (a plurality of) sensors which are provided and designed to acquire data of process steps of the sterile product cycle.

In a generic device this object is consequently solved in accordance with the invention preferably in that a holding device is provided for a/in a sterile product, in particular for a/in a sterile product container, for a/in a sterile product screen, or for a/in a soft packaging. The holding device is provided to hold a medical product to be sterilised in/at the holder and/or in the sterile product screen during an (entire) preparation process/cycle. The holding device comprises communication means. A part of the communication means, for instance, the communication module as described further below, is designed for a frequency range such that the part, when the sterile container is closed, supplies data in relation to the medical product to an environment externally of the sterile product container.

This has the advantage that the monitoring of the entire sterile product cycle is improved.

As an alternative or in supplement to the sterile product container a soft packaging may be used. The soft packaging may be a screen, wrapped by a specific paper. Here, the screen forms the holding device.

The sterile product container may mean a sterile product container with an inserted screen basket, for instance, the sterile product screen. The sterile product screen may mean a screen basket in soft packaging.

Specifically, frequencies may be provided which can penetrate an all-metal container as a sterile product container in a lossy manner. The sterile product container may be hermetically sealed, for instance, during use. Specific frequencies may, for instance, be 2.4 GHz and 5 GHz. For Bluetooth, in particular a frequency range from 2.402 GHz to 2.480 GHz may be provided. For WLAN, in particular a frequency range from 2.412 GHz to 2.484 GHz and/or from 5.18 GHz to 5.825 GHz may be provided. This may correspond to the standards 802.11 b/g/n or 802.11 a/h/j/n/ac.

The communication means have to be interpreted broadly and may also comprise sensors, as described further below. Any form of communication and/or interface may be comprised here. Specifically, the communication means may transmit receiving signals/transmitting signals/sensor signals to a unit connected in terms of communication. This connected unit may be the evaluation unit/data processing unit described further below.

Advantageous embodiments are claimed in the subclaims and will be explained in detail in the following.

The holding device may in particular be a screen (within) the sterile product/sterile product container, for instance, the sterile product screen. Thus, the screen may contain the individual elements, for instance, the communication means. The screen may be in the form of a basket, i.e., a screen basket. This screen basket may contain holders for insertion in the sterile product/the sterile product container and may otherwise comprise close meshed grids in order that medical products to be sterilised are held but cannot fall or slip through the grids.

The environment externally of the sterile product container (container) may in particular be a relatively close environment. The environment is, for instance, in a range of maximally 8 meters or maximally 4 meters. In other words, the environment corresponds to the afore-mentioned range (within which a receiving unit has to be located). It may also be sufficient if the environment is merely defined as a 1 meter radius around the sterile product (the sterile container/screen). A signal intensity of the corresponding communication means may be adapted accordingly.

The holding device may comprise a data processing device and an energy supply unit. The data processing device is, for instance, designed to minimise an energy supply provided by the energy supply unit for the communication means of the holding device associated with the data processing device between the process steps of the preparation process.

The data processing device and/or the communication means may further comprise an insulation surrounding them jointly or individually and insulating thermally and/or water-tightly.

Moreover, the communication means may comprise an NFC read and write device which is mounted on the holding device such that, during insertion of the medical product to be sterilised in the holding device, an NFC communication with a complementary NFC antenna of the medical product to be sterilised is enabled.

The holding device may be of modular structure, so that the removing of individual elements of the holding device is enabled.

The above-defined object is also solved by a container, such as a sterile product container or a soft packaging. The sterile product container, but possibly also the soft packaging, forms a Faraday cage of metal in a closed state. The metal may be a non-ferromagnetic metal. A metal layer of the sterile product container may be a sheet metal, wherein it is (distinctly) smaller than the penetration depth of the currents induced therein by an alternating field between an inner-side transponder and an outer-side receiver.

In an advantageous embodiment of the disclosure, which may be designed in combination with other features or else individually, it is provided that the sensors are designed redundantly.

In an advantageous embodiment of the disclosure, which may be designed in combination with other features or else individually, it is provided that the holding device comprises a plurality of antennas and a read and write unit, wherein the antennas are connected to the read and write unit via an electronic distribution system (multiplexer).

In an advantageous embodiment of the disclosure, which may be designed in combination with other features or else individually, it is provided that the holding device is of modular structure and enables the removing of individual elements of the holding device.

In an advantageous embodiment of the disclosure, which may be designed in combination with other features or else individually, it is provided that the evaluation unit compares the individual process steps with predetermined parameters and increments a respective counter when the evaluation unit determines that the process step was performed successfully.

In an advantageous embodiment of the disclosure, which may be designed in combination with other features or else individually, it is provided that the holding device (1) comprises a signal transmitter indicating information about the process steps to a user by an optical, acoustic, and/or haptic signal.

In an advantageous embodiment of the disclosure, which may be designed in combination with other features or else individually, it is provided that the signal transmitter indicates a position of a medical product to the user by the optical, acoustic, and/or haptic signal.

The above-defined object is also solved by a method for monitoring a sterile product cycle. The method comprises
providing a holding device in a sterile product case, such as a sterile product container, a sterile product screen, or a soft packaging,
providing a medical product to be sterilised in the sterile product container and closing same in an all-metal manner,
performing a sterile cleaning in the sterile product container,
during performing of the sterile cleaning, monitoring the medical product by means of the parameter data representative of the sterile cleaning which are acquired by at least one sensor of the plurality of sensors at the holding device/holder.

Likewise, the holding device may be inserted in a cleaning bath, in particular in the form of a screen basket during the preparation, for instance, ultrasonic cleaning, wherein at least one sensor of the plurality of sensors at the holding device acquires parameter data representative of the ultrasonic cleaning.

Likewise, during maintenance with oil, the products may be left on the holder, wherein at least one sensor of the plurality of sensors at the holding device acquires parameter data representative of the oil maintenance. This also applies for the cleaning process, wherein at least one sensor of the plurality of sensors at the holding device acquires parameter data representative of the cleaning process. During the maintenance process with oil the oil bottle to be used should stand or be held vertically so as to ensure an optimum oil flow. This may, for instance, be ensured by an inclination sensor in/at the holding device. Thus, it is possible to ensure and trace the correct position of the oil bottle during the maintenance process.

In other words, the disclosure preferably relates to the monitoring of a sterile product cycle, in particular the recognition of ultrasonic cleanings, tracking in the closed system, retrievable via Bluetooth and/or WiFi, environment sensors, automatic recognition of process steps and/or monitoring of products.

For instance, a device (sensor) for the recognition of temperatures, for the recognition of preparation processes, which include cleaning, disinfection, maintenance and/or sterilisation, may be provided. Furthermore, a method of equipping products with wireless identification markers (transponders) may be described herein. Th data may also be stored in a server or cloud solution. The transponders may be read and/or written by means of the holding device and/or its elements for medical products described herein.

The disclosure may allow a comprehensive monitoring of the entire sterile product cycle of products. For this purpose, various sensors at the one holding device can automatically recognize different process steps, evaluate the data autonomously by means of an evaluation device of the one holding device, change the corresponding operating mode, and/or store the results, on the one hand, and write them on a product transponder, on the other hand. Furthermore, the reading out of transponders and sensor data may be implementable by an intelligent “module” which is installed at a/the holder/holding device (also to be referred to as a tray), for instance, at/in a screen basket and/or a sterile container. In order to signal important information to the user, optical, acoustic, or haptic signal transmitters may be used.

Moreover, the disclosure may relate to the complete detection and analysis of the sterile product cycle (use in the operating room, ultrasonic cleaning, cleaning in the cleaning and disinfecting device, maintenance, e.g. by means of oil spray and sterilisation) and to the providing of detailed data about the corresponding individual product. The construction of the system and/or of the device may either be designed as a holder of its own, in accordance with the holding device described here, or as an insert for a screen basket, also as the holding device described here.

The sensors of the holding device acquire in their entirety data concerning a use in the operating room, ultrasonic cleaning, cleaning in the cleaning and disinfecting device, maintenance, e.g. by means of oil spray and sterilisation.

The positioning of the electronics may be relatively freely selectable (e.g. inside or below a screen basket) since, due to the frequency range used, no shielding by the metal environment occurs. Preferably, however, the electronics is connected firmly to the holder, in particular the screen (not, however, to the container).

For the use of a plurality of antenna systems slots may be incorporated by which the plurality of antennas can be connected to a read and write unit via an electronic distribution system (multiplexer).

The system may further comprise a data evaluation unit (microcontroller), a communication module (e.g. Bluetooth or Wi-Fi in the range of 2.4 to 5.825 GHz), an exchangeable energy supply (e.g. high-temperature batteries, power cap), an RFID/NFC module (read and write module), and/or various sensors (temperature, air moisture, pH value, pressure, sound waves, flow rate, voltages, light or vibrations) and signal transmitters (acoustic, optical, or haptic). Energy harvesting may also be used a partial energy supply at least.

The system may further be of modular structure and may be constructed in line with the given requirements as requested. For the protection of the electronics prior to cleaning and sterilisation a thermal and shielding (from moisture and chemicals) insulation may be used. The positioning of the sensors may be such that the individual process steps can be detected safely. The energy supply may be exchanged either after the end of the lifetime or be recharged by a predetermined changing interval at defined process places.

One of the aspects may comprise a smart tray or a smart screen. It may not only be the matter of a holder as such, but of an entire screen.

A further aspect may be a detection of the ultrasonic cleaning, which may be performed by means of a suitable sensor system.

Yet a further aspect may be the locating of products. Products may be tracked and recognized through closed sterile product containers and screen baskets (Faraday cage) via Bluetooth or Bluetooth Low Energy, BLE, and/or W-LAN. A signal may be output by the smart tray “module” which then outputs a sound or vibrates. A great advantage is that the container need not be opened to find a product. If a container is opened, it is deemed to be unsterile if it is not used, and it is supplied to the preparation process again. This results in high costs which can thus be avoided.

Yet a further aspect may relate to data transmission through the closed sterile product container by means of Bluetooth and W-LAN.

The invention may differ from the state of the art such that a complete monitoring of the sterile product cycle can be detected. The automatic recognition and analysis of the process steps can allow an evaluation of the entire process and enables calculations for lifetimes, damages, and usage behaviour of individual products. A further improvement may be a detachment from a specific holding device which is designed for a product type to a solution in which a screen basket is modified such that it can serve as a system for the evaluation of sensor data and the reading/writing of transponders.

A further difference may be that an automatic recognition of ultrasonic cleaning, which is very important for the hygiene of the medical products, is provided.

A focus may in particular be directed to the detection of the entire sterile product cycle. Moreover, the use of ultrasonic sensors (e.g. piezo element, radiometer, microphone) may be provided for the recognition of pre-cleaning by an ultrasonic bath.

An important process step of the sterile product cycle is ultrasonic cleaning. It may be detected by ultrasonic sensors whether ultrasonic cleaning was performed. Ultrasonic sensors are, for instance, a piezo element, a radiometer, and/or a microphone, whereby multiple redundancy is achieved.

An extension of the usable sensors to (temperature, pressure) air moisture, pH value, sound waves, flow rate, voltages, light, and/or vibrations may also be provided.

In an advantageous embodiment of the disclosure, which may be designed in combination with other features or else individually, it is provided that the sensors detect temperature, air moisture, pH value, pressure, sound waves, voltages, and/or vibrations.

Moreover, an extension of the holding system to a screen basket comprising connections for antennas and an evaluation unit may be provided.

Likewise, a free positioning of the electronics by communication in the GHz frequency range (no shielding) in the container or screen basket (Faraday cage) may be provided.

For example, the use of Bluetooth or WiFi (2.4-5.825 GHz) may be provided for the tracking of the system.

Moreover, locating of products through the closed sterile container and through closed screen baskets (Faraday cage) may be performed.

Furthermore, data transmission may take place through the closed container by means of Bluetooth and W-LAN.

The disclosure may, for instance, implement a continuous monitoring of the medical products. By means of the data obtained it may be possible to establish calculations about the lifetime and the condition of the medical products. Furthermore, the individual process steps can be recognized autonomously by the device. By the providing of additional data it is possible to implement a calculation of possible damages. By signal transmitters, for instance, the user may be informed about the position, the condition, and the process step. The user can localise and identify products through closed sterile containers without having to open the containers, which would result in high reprocessing costs. The customer can localise and identify products through closed soft packaging without having to open the soft packaging, which would result in high reprocessing costs.

In an advantageous embodiment of the disclosure, which may be designed in combination with other features or else individually, it is provided that the holding device comprises an evaluation unit that infers the lifetime and/or a condition of the medical products from the data.

An exemplary case for the disclosure will be explained in the following and is a holding device for medical motor systems. They are equipped with NFC transponders which comprise a unique identification number matching with the motor systems. Furthermore, the transponders are designed such that information about cycle numbers of pre-cleaning, cleaning, maintenance, sterilisation, and use (e.g. duration of current feed, etc.) can be stored optionally together with detailed data.

The holding device is, for instance, designed such that sensors can be placed together with a protected evaluation unit and an exchangeable energy supply. The entire electronics is, for instance, designed to be exchangeable. Sensors for detecting the temperature, air moisture, pH value, pressure, sound waves, voltages, position, and/or vibrations can be used. For the reading and writing of the NFC transponders, for instance, at least one antenna is connected to the evaluation unit and mounted such that the transponders are within the effective range at any time.

The sensors for detecting the process steps are, for instance, mounted such that an optimal detection can take place without disturbing the operation, the cleaning effect, or the handling. In order that the energy requirement of the module is as small as possible, only those systems are activated, for instance, which are needed. Between two process steps the module is, for instance, in an energy saving mode where checking of whether a process step has been initiated takes place at defined intervals. Examples of this are temperature limits prior to cleaning, maintenance, and sterilisation, as well as a change of light during opening before or the use of ultrasonic waves during pre-cleaning.

For the detection of ultrasonic cleaning, for instance, a piezo element may be used. The recognition of use and the information about the operating mode can take place by means of the control device provided, which stores the data on the individual handpiece.

For the detection of cleaning, maintenance with oil spray, and sterilisation, a pair of temperature sensors can be used in a basic variant. The sensors can be designed redundantly in any case and/or be examined automatically by means of a self-check.

The evaluation unit analyses, for instance, the individual process steps and compares them with predetermined parameters. If they have been achieved with acceptable deviations, the respective internal counter is incremented. The updated counter value and/or the process data are subsequently transferred to the NFC transponders. By means of a signal transmitter which is capable of emitting optical, acoustic or haptic signals the user is informed about the process steps and the correct duration of the oil spraying process.

A further use of the signal transmitter is, for instance, the enabling of localising of the associated medical product within a rigid or soft sterile barrier system (e.g. sterile container, screen, soft packaging). Here, the user can activate a search function by means of Bluetooth or Wi-Fi.

Subsequently, the corresponding signal transmitter is activated, so that the position of the product is indicated by a signal (sound, light, vibration) in the closer environment. A further possibility is, for instance, localising by means of triangulation or signal intensity measurement, which are possible with a radio standard (e.g. Bluetooth, Wi-Fi).

A first evaluation of the sensor values is, for instance, performed by the integrated evaluation unit, whereby processes can be analysed. For a calculation of lifetimes, possible damages, and further product-related predictions (predictive maintenance), the data are, for instance, analysed additionally in an external evaluation unit (e.g. a PC, tablet) or a server-based solution (e.g. cloud, server).

In one example the disclosure thus relates to a holding system capable of detecting the complete sterile product cycle of a medical product (e.g. surgical instrument, implant, etc.) and of providing detailed sensor data offline and online. A detection of ultrasonic pre-cleaning is performed by means of sensors and thus enlarges the data available. Furthermore, a product locating system is, for instance, integrated in the screen and is thus an integral component of this disclosure in the closed container.

Additional embodiments will be described in the following.

The holding device may be provided for holding at least one sterilisable medical product in particular during a preparation process and for storing the medical product before and/or after the preparation process. The data processing device can be connected/connectable in a signal-conducting manner to at least one sensor. The sensor may in particular be a temperature sensor for determination of a temperature during a preparation process and/or a pressure sensor for determination of a static and/or dynamic pressure of a cleaning liquid during the preparation process.

The data processing device processes, for instance, the sensor signals to cleaning information and comprises a storage device and/or is connected/connectable in a data-conducting manner to a storage device on which the cleaning information is stored/is storable. The cleaning information is retrievable/readable from the storage device by an evaluation unit, so that, by means of the cleaning information, a cleaning profile of the medical product can be determined by the evaluation unit.

Accordingly it is possible to document the preparation process of the medical product by means of the cleaning profile without the volume and/or the weight of the medical product being changed. It is specifically also provided that histories of the sensor data are to be constituents of the cleaning profile. The signal-conducting connection between the data processing device and the sensor can, for instance, be established by means of a wired connection and/or a wireless connection.

Apart from the determination of the pressure of the cleaning agent and the temperature it is also provided that, for instance, an amount of cleaning oil, a pH value, a conductivity or the like can be determined by means of one or a plurality of sensors. It is provided that histories, cycles, holding times, time lapses or the like can also be detected for all the data determined.

By way of example it is provided that the data processing device may, for instance, also be a so-called SOC (system on a chip). In order to be able to use the data processing device at the holder, i.e. within an area to be cleaned and/or to be sterilised, it is provided that the data processing device is protected from environmental influences.

The evaluation unit may, for instance, be a PC, a laptop, a tablet or the like. Moreover, it is also possible that the evaluation unit can be designed as a constituent of a further computer system, for instance, a control device of the medical product.

In one embodiment it is provided that the holding device comprises a feed line and at least one cleaning line branching off the feed line and to which a rinsing intake for receiving the medical product in particular of a handpiece is connected, wherein, through the feed line, the cleaning line, and the rinsing intake, the cleaning agent can be introduced into sections to be cleaned in particular of the handpiece. Handpieces may, for instance, mean surgical instruments with internal or external drives as well as with intakes for surgical tools.

It is of advantage that the holding device may comprise a plurality of cleaning lines, wherein each cleaning line is assigned to a rinsing intake, so that, by monitoring the cleaning line, it can be inferred whether a preparation process has been performed in accordance with predetermined parameters and the medical products arranged at the rinsing intakes, in particular handpieces, have been cleaned adequately.

A temperature sensor and/or a pressure sensor is, for instance, arranged in the feed line and/or the cleaning line and/or the rinsing intake. Due to a thermodynamic and hydraulic coupling between the feed line, the cleaning lines, and the rinsing intake it is sufficient for determining the cleaning profile if each cleaning line comprises a pressure sensor and the feed line comprises a temperature sensor.

The determined temperature in the feed line and the pressure in the cleaning lines during the cleaning process define the parameters by means of which it can be determined that the cleaning process was run through by every medical product, in particular handpiece, in a form guaranteeing an appropriate and hygienic preparation.

In order to achieve a simple construction of the holding device it is also possible and provided that merely the feed line may comprise both a pressure sensor and a temperature sensor. This is in particular of advantage if predominantly similar medical products in particular handpieces with similar degrees of contamination are to be cleaned.

In one embodiment it is provided that the data processing device is connected/connectable to a data storage of the medical product, wherein the cleaning information is stored/is storable in the data storage. Accordingly it is provided that the data processing device writes the cleaning information determined into the data storage of the medical product. In the case of handpieces which are connected to an associated control device prior to their use it is provided that the stored cleaning information can be read by the control device and/or be displayed. Such data transfer may, for instance, take place via a wired connection or via radio.

In the case of such an embodiment of the holding device a linking of the cleaning information with an identity of the medical product takes place by the cleaning information assigned to the medical product being written directly into the data storage of the medical product.

It is, for instance, provided that the holding device comprises at least one identification arrangement brought/adapted to be brought into operative connection with the data processing device, and by means of which the medical product arranged in the holding device can be identified on the basis of an identification feature, wherein, by means of the data processing device, the identification feature can be linked with the cleaning information to product information of the medical product. Such an embodiment of the disclosure enables that the cleaning information processed to product information can be transferred independently of the medical product and are thus suited to be used also in the scope of an external analysis, during stock control, during the monitoring of service intervals, or the like.

It is, for instance, further provided that the identification arrangement may, for example, be a reading device for reading an NFC chip, wherein the identification feature is an NFC chip arranged at the medical product. Identification feature in accordance with the inventive concept means any means as well as identification information stored therein, which are suited to identify an object by means of an automatable method. When linking the cleaning information to the identification feature, the cleaning information is enhanced by the identification information of the identification feature. Identification features are in particular NFC chips, resistance encodings, EEPROMs, barcodes, machine-readable numerical sequences, or the like. It is also possible that the identification arrangement is integrated partially or completely in the data processing device.

By way of example, the identification arrangement can be brought into operative connection with the data processing device both by means of a wire and wirelessly. It is provided that the wireless connection can be established, for instance, by means of a radio standard, e.g. by WLAN.

Another example provides that an identification of the medical product takes place at least partially by means of a wireless method. Identification of the medical product in accordance with the inventive concept means in particular which combination of identification arrangement and identification feature is used to identify the medical product. Wireless methods comprise both radio-based electronic methods and, for instance, optical methods. Radio-based electronic methods are, for instance, the use of RFID technology, WLAN, NFC, or the like. Optical methods comprise, for instance, the use of QR codes, barcodes, machine-readable numerical sequences, or the like.

It is also possible that an identification of the medical product takes place at least partially by means of a wired method. In the wired methods it is provided that a physical connection, in particular a wire connection, is established between the identification arrangement and the identification feature. Identification features which are in particular suited for a wired identification are, for instance, resistance encodings, EEPROMs, or the like.

It is moreover possible that different identification means can be used in parallel so as to achieve a redundancy when identifying the medical product. This reduces in particular an error rate during the identification of the medical product and increases fail-safety.

In order to achieve a direct transmission of the cleaning information and/or product information from the data processing device to the evaluation unit it is possibly provided in accordance with the invention that the data processing device is brought/is adapted to be brought into operative connection with a transmission device, for instance, a part of the communication means, in a data-conducting manner, wherein the transmission device establishes/is adapted to establish a data-conducting connection between the data processing device and the evaluation unit, by means of which cleaning information and/or product information is transmissible from the data processing device to the evaluation unit.

Such an embodiment of the holding device is particularly advantageous when the medical products to be cleaned do not have their own data storage. Such medical products are, for instance, compressed air handpieces, re-processable implants, tools, instruments, or the like. It is provided that the evaluation unit may, for instance, be a PC, a tablet, a smartphone, or the like. It is particularly advantageous in this embodiment that, for instance, the content of a sterile screen, tray, soft packaging, and/or sterile product container can be examined without it having to be opened and its seal having to be broken.

Alternatively it may be provided that the transmission device is designed such that a data-conducting connection with the evaluation unit can be established by means of a radio standard, in particular a mobile communication standard, a WLAN standard, and/or a near field communication standard. By the use of a radio standard the connection of the evaluation unit to the data processing device by means of the transmission device is facilitated since standardised protocols can be resorted to. Contrary to this it is, however, also possible that a generic radio standard can be used for establishing the connection.

It is provided that the product information between the holding device and the evaluation unit can be transmissible both directly and indirectly. A direct connection can be designed both wirelessly and wired. It is provided that an indirect connection can be established in particular via the data storage of the medical product, namely in that the data storage is first connected to the holding device and the product information is stored on the data storage, and subsequently the medical product is connected along with the data storage to the evaluation unit, so that the product information can be read out from the data storage by the evaluation unit.

In order to enable an assignment of the product information to the correct medical product by the evaluation unit it is provided that the evaluation unit may comprise means for reading out information features or product information stored in the data storage of the medical product.

The evaluation unit can be brought/be adapted to be brought into operative connection with an external storage medium, wherein the cleaning information and/or the product information can be transmitted from the data processing device to the external storage medium via the evaluation unit. As a result of this, cleaning information can be exported and be evaluated and/or stored on external systems. Thus, measures for quality assurance and for hygienic monitoring with respect to the medical products are facilitated.

The external storage medium may be a cloud storage. By the storing of the cleaning information in a cloud storage it is enabled that a responsible group of people, in particular surgeons, personnel of the central sterile product supply department, the manufacturer, and the like, can obtain information about a condition of the medical products independently of the place. Moreover, it is possible for the manufacturer of the medical products that product updates, recall actions, or the like can be centralised and performed and/or coordinated with less organisation effort.

It is clear for the person skilled in the art that the explanations made here may be/can be implemented with the use of hardware circuits, software means, or a combination thereof.

The software means may be in connection with programmed microprocessors or a general computer, an ASIC (Application Specific Integrated Circuit), and/or DSPs (Digital Signal Processors).

The holding device, the sterile product/the sterile container and/or their (structural) elements may, for instance, be implemented as a computer, a logic circuit, an FPGA (Field Programmable Gate Array), a processor (comprising, for instance, a microprocessor, a microcontroller (pC) or a vector processor)/Core (may be integrated in the processor or be used by the processor, respectively)/CPU (Central Processing Unit; wherein a plurality of processor cores are possible), an FPU (Floating Point Unit), an NPU (Numeric Processing Unit), an ALU (Arithmetic Logical Unit), a coprocessor (additional microprocessor for support of a central processing unit (CPU)), a GPGPU (General Purpose Computation on Graphics Processing Unit), a parallel computer (for simultaneous execution, inter alia on a plurality of central processing units and/or graphic processors, of computing operations), or a DSP.

It is moreover clear for the person skilled in the art that, also when the details described here are described with respect to a method, these details may also be implemented in a suitable device, a computer processor, or a storage connected to a processor, wherein the storage is provided with one or a plurality of programs performing the method when executed by the processor. Methods such as swapping and paging may be used.

Even if some of the afore-described aspects have been described with respect to the holding device, these aspects may also apply for the sterile product/the sterile product container and the method. Likewise, the aspects described before with respect to the sterile good/the sterile good container may correspondingly apply for the method and the holding device and screens.

If it reads here that a component is “connected” to another component, is “linked to it” or “accesses it”, this may mean that it is directly connected to it or accesses it directly; however, it has to be noted that another component may be interposed. If it reads, on the other hand, that a component is “directly connected” to another component or “accesses it directly”, this means that no further components are available therebetween.

### BRIEF DESCRIPTION OF THE FIGURES

The disclosure will be explained in the following by means of drawings. There show:

FIG. 1 a schematic illustration of a holding system with its elements; and

FIG. 2 a schematic illustration of a system process.

The Figures are merely of schematic nature and serve exclusively for the understanding of the invention. The same elements are provided with the same reference numbers. The features of the individual embodiments may be exchanged mutually.

Moreover, spatially relative terms such as “positioned below”, “under”, “lower”, “positioned above”, “upper”, “left”, “right”, and the like may be used for the simple description of the relation of one element or one structure to one or a plurality of other elements or structures illustrated in the Figures. The spatially relative terms shall, in addition to the orientation illustrated in the Figures, comprise other orientations of the component in use or in operation. The component may be oriented differently (turned by 90 degrees or in another orientation), and the spatially relative descriptors used here may also be interpreted correspondingly.

### DESCRIPTION OF THE FIGURES

The holding device, the method, and the sterile product, i.e., the sterile product container or sterile product screen, will now be described by means of embodiments.

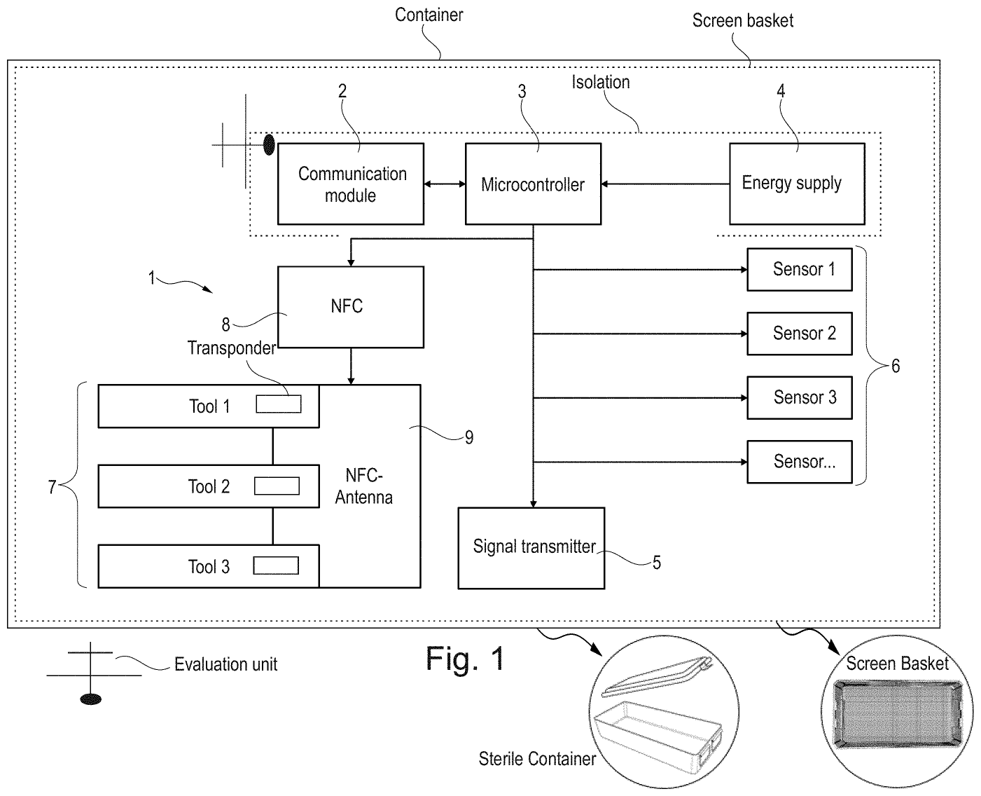

FIG. 1 shows a schematic illustration of a holding system 1 with its elements. Specifically, the holding system 1 comprises a communication module 2 provided for the external communication of data. For this purpose, the data are provided by a microcontroller 3 which is in direct communication with the communication module 2. The microcontroller 3 controls an energy supply provided by an energy supply unit 4. For this purpose, the microcontroller 3 can influence the energy supply such that the energy supply is phased down and/or placed in an energy saving mode between process steps.

Furthermore, the microcontroller 3 is in direct communication with sensors 6. The sensors may be of different kind and be one or a plurality of the kind mentioned here. Likewise, the microcontroller 3 may be in contact with a signal transmitter 5. For this purpose, the sensors 6 may be in direct or indirect contact with the signal transmitter 5 via the microcontroller 3. The signal transmitter 5, for instance, an LED, a loudspeaker, or a radio module, informs the user, for instance, about where a medical product is just being and/or at which process step the system 1 is at the moment. The system 1 further comprises an NFC read and (possibly) write device 8 which is in connection and/or communicates in operation with one or a plurality of NFC antennas 9 of different products 7. This may take place by directly placing the products 7 on the NFC read and write device 8 and/or a plurality thereof.

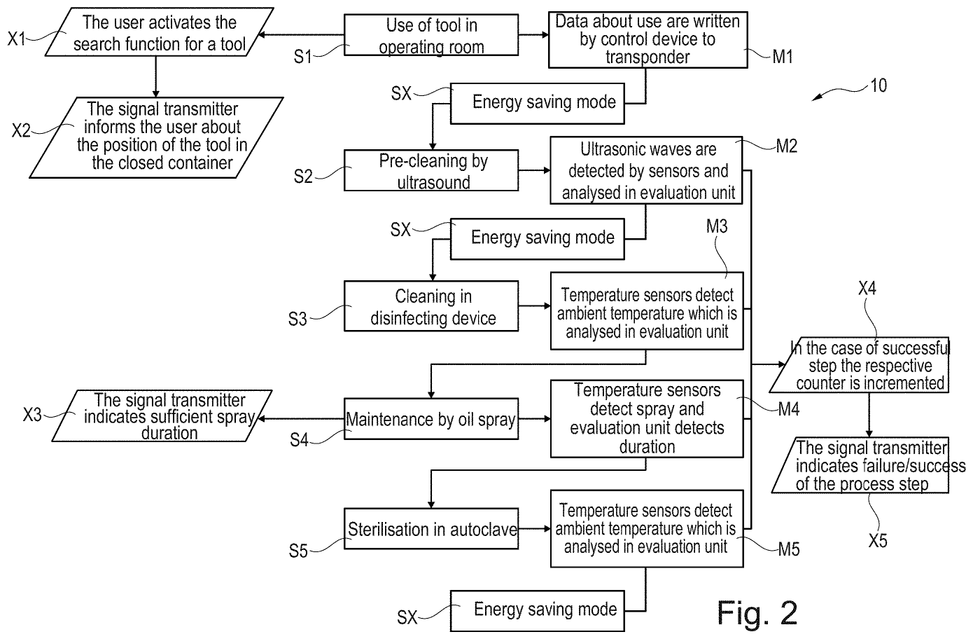

Further details and aspects are mentioned in connection with the embodiments described before or in the following. The embodiment shown in FIG. 1 may comprise one or a plurality of optional additional features which correspond to one or a plurality of aspects mentioned in connection with the proposed concept or the embodiment described in the following with reference to FIG. 2.

FIG. 2 shows a schematic illustration of a possible system process 10. The system process 10 may, for instance, start with Step S1 in which the product is used during an operation. The user may then activate a search function for this product in Step X1. The signal transmitter 5 indicates the position of the product e.g. in the closed container to the user, see X2. The data about the use are written by the control device/microcontroller 3 onto the transponder, see M1. Then, the system 1 enters the energy saving mode Sx.

After termination of the energy saving mode Sx the pre-cleaning by means of ultrasound starts, see S2. The ultrasonic waves are detected by sensors 6 and analysed in the evaluation unit, for instance, after communication via the communication module 2 to outside of the holder (i.e., the container or the soft packaging), see M2. Then, the system 1 again enters the energy saving mode Sx.

After termination of the energy saving mode Sx the cleaning in the cleaning and disinfecting device starts, see S3. In this process the temperature sensor 6 detects the ambient temperature (and/or inner temperature of the holder) which is analysed in the evaluation unit 3, see M3. Then, maintenance by means of an oil spray is performed, see S4. The signal transmitter 5 indicates the sufficient duration of the spray, see X3. The temperature sensors 6 detect the spray and the evaluation unit detects the duration, see M4. After that, sterilisation takes place in the autoclave (not illustrated), see S5. The temperature sensors detect the ambient temperature (and/or the inner temperature of the holder and/or container) which is analysed in the evaluation unit, see M5. Then, the system 1 enters the energy saving mode again, see Sx.

In the case of successful steps M1, M2, M3, or M4 the respective counter is incremented, see X4, and/or the signal transmitter 5 indicates the failure/success of the process step, see X5.

Further details and aspects are mentioned in connection with the embodiments described before or in the following. The embodiment shown in FIG. 2 may comprise one or a plurality of optional additional features which correspond to one or a plurality of aspects mentioned in connection with the proposed concept or one or a plurality of embodiments described before (e.g. FIG. 1).

### LIST OF REFERENCE SIGNS

1 holding system
2 communication module
3 microcontroller
4 energy supply
5 signal transmitter
6 sensors
7 products
8 NFC read and write device
9 NFC antenna
10 method
S1 . . . S5, Sx operations
X1 . . . X5 events
M1 . . . M5 sensor events

1. A holding device for medical products mounted on the holding device during a sterile product cycle, the holding device comprising:
a plurality of sensors which are provided and configured to acquire parameters of preparation process steps, wherein each individual sensor is configured to acquire a different respective data parameter, and wherein respective data parameter is representative of a respective individual process step of the sterile product cycle differing from each other respective individual process step; and
an evaluation unit configured to analyse the respective data parameters, on the basis of which an identification of a one of the respective individual process steps just performed takes place.

2. The holding device according to claim 1, wherein the plurality of sensors are configured to acquire in their entirety parameter data with respect to a use of the medical products in an operating room, and/or an ultrasonic cleaning, and/or a cleaning in a cleaning and disinfecting device, and/or a maintenance by lubricating, and/or a sterilisation.

3. The holding device according to claim 1, wherein the sensors are configured to detect in their entirety temperature, air moisture, pH value, pressure, sound waves, electric voltages, and/or vibrations.

4. The holding device according to claim 3, wherein at least one sensor of the plurality of sensors is a piezo element.

5. The holding device according to claim 3, wherein at least one sensor of the plurality of sensors is a radiometer.

6. The holding device according to claim 3, wherein at least one sensor of the plurality of sensors is a microphone.

7. The holding device according to claim 1, wherein the evaluation unit is configured to infer a lifetime and/or a condition of the medical products from the parameter data.

8. The holding device according to claim 1, wherein the plurality of sensors comprise at least two redundant sensors.

9. The holding device according to claim 1, wherein the holding device comprises a plurality of antennas and a read and write unit, wherein the antennas are connected to the read and write unit via an electronic distribution system (multiplexer).

10. The holding device according to claim 1, wherein the holding device comprises a modular structure configured to enable a removing of individual elements of the holding device.

11. The holding device according to claim 7, wherein the evaluation unit is configured to compare the respective data parameters representative of respective individual process steps with predetermined parameters, and to increment a respective counter when the evaluation unit determines that an individual process step was performed successfully.

12. The holding device according to claim 1, wherein the holding device comprises a signal transmitter configured to indicate information about one or more of the individual process steps to a user by an optical, acoustic, and/or haptic signal.

13. The holding device according to claim 1, wherein the holding device comprises a signal transmitter configured to indicate a position of the medical product to a user by an optical, acoustic, and/or haptic signal.

14. A holding device system comprising:
a medical device holder;
a plurality of sensors provided on the medical device holder; and
a processor provided on the medical device holder and in communication with the plurality of sensors, wherein the processor is configured to:
a) receive a respective signal from each of the plurality of sensors, each respective signal representing a respective measured parameter of at least one of a plurality of process steps performed on the holding device system, wherein at least two of the respective measured parameters are different from each other, and
b) analyse the measured parameters to determine an identity of at least one of the plurality of process steps.

15. The holding device system of claim 14, wherein the plurality of process steps comprise one or more of: use of a medical product in an operating room; an ultrasonic cleaning; a cleaning in a cleaning and disinfecting device; a maintenance by lubricating; and a sterilisation.

16. The holding device system of claim 14, wherein the plurality of sensors are configured to detect one or more of: temperature; air moisture; pH value; pressure; sound waves; electric voltages; and vibrations.

17. The holding device system of claim 16, wherein the plurality of sensors comprise at least one of: a piezo element; a radiometer; and a microphone.

18. The holding device system of claim 14, further comprising one or more antennas and a read and write unit, wherein the one or more antennas are connected to the read and write unit via an electronic distribution system.

19. The holding device system of claim 14, further comprising a signal transmitter configured to indicate information about one or more of the process steps to a user by an optical, acoustic and/or haptic signal.

20. The holding device system of claim 14, further comprising a signal transmitter configured to indicate a position of a medical device to a user by an optical, acoustic and/or haptic signal.
